(19) United States
(12) Patent Application Publication
(10) Pub. No.: US 2017/0309086 A1
(43) Pub. Date: Oct. 26, 2017

(54) METHOD AND APPARATUS FOR RESCUING DRIVERLESS VEHICLES

(71) Applicant: Baidu Online Network Technology (Beijing) Co., Ltd., Beijing (CN)

(72) Inventors: Hong ZHAI, Beijing (CN); Yi WU, Beijing (CN)

(21) Appl. No.: 15/279,932

(22) Filed: Sep. 29, 2016

(30) Foreign Application Priority Data
Apr. 22, 2016 (CN) 201610256746.8

(51) Int. Cl. G07C 5/00; G07C 5/02; G05D 1/00; A61B 5/18; A61B 5/00; A61B 5/021; G07C 5/08

(52) U.S. Cl. CPC G07C 5/008; G07C 5/0808; G07C 5/02; G05D 1/0088; A61B 5/6893; A61B 5/0013; A61B 5/021; A61B 5/0077; A61B 5/0022; A61B 5/18

(57) ABSTRACT

The present application discloses at least a method and apparatus for rescuing driverless vehicles. A specific implementation of the method includes: collecting status information of a driverless vehicle; determining whether the driverless vehicle has an autonomous driving ability; if not, setting a driving mode of the driverless vehicle to a manual driving mode and further determining whether the driverless vehicle has a traveling ability in the manual driving mode; and collecting passenger information of the driverless vehicle and sending the status information and the passenger information to a cloud server providing support for the driverless vehicle, in response to the driverless vehicle not having the traveling ability in the manual driving mode. This implementation may achieve effective vehicle rescue.

### CROSS REFERENCE TO RELATED APPLICATIONS

This application claims the priority of Chinese Patent Application No. 201610256746.8, entitled “Method and Apparatus for Rescuing Driverless Vehicles,” filed on Apr. 22, 2016, the content of which is incorporated herein by reference in its entirety.

### TECHNICAL FIELD

The present application relates to the field of driverless vehicle technology, specifically to the field of driverless vehicle networking technology, and more specifically to a method and apparatus for rescuing driverless vehicles.

### BACKGROUND

A driverless vehicle is a novel intelligent automobile, also called a “wheeled mobile robot.” The driverless vehicle mainly uses an electronic control unit (ECU) (that is, a vehicle-mounted intelligent device) to perform precise control, computation and analysis for various parts in the vehicle, so that fully automatic driving of the vehicle operates is realized, thereby achieving autonomous driving of the vehicle.

Existing vehicle rescue technologies generally collect information from a vehicle on the premise that the vehicle is in a manual driving mode. However, with driverless vehicle technologies, such rescue technologies cannot accurately determine whether the driverless vehicle has a capability to drive autonomously, and therefore cannot efficiently rescue driverless vehicles that break down.

### SUMMARY

An objective of the present application is to provide an improved method and apparatus for rescuing driverless vehicles, so as to solve the technical problem mentioned in the foregoing Background section.

According to a first aspect, the present application provides a method for rescuing driverless vehicles, comprising: collecting status information of a driverless vehicle; determining whether the driverless vehicle has an autonomous driving ability; if not, setting a driving mode of the driverless vehicle to a manual driving mode and further determining whether the driverless vehicle has a traveling ability in the manual driving mode; and collecting passenger information of the driverless vehicle and sending the status information and the passenger information to a cloud server providing support for the driverless vehicle, in response to the driverless vehicle not having the traveling ability in the manual driving mode.

In some embodiments, determining whether the driverless vehicle has the autonomous driving ability comprises: determining that the driverless vehicle has the autonomous driving ability when following two conditions are both satisfied: the driverless vehicle has an ability of switching to an autonomous driving mode or the driverless vehicle is in the autonomous driving mode; and a sensor related to the autonomous driving mode and a circuit connected to the sensor are in a normal operating condition.

In some embodiments, collecting status information of the driverless vehicle comprises: collecting the status information of the driverless vehicle in real time or periodically.

In some embodiments, the sensor comprises at least one of: a sensor for detecting an operating status of a brake; a sensor for detecting an operating status of a vehicle-mounted battery; a camera, a sensor for detecting a fuel status; a sensor for detecting an air status inside the vehicle; and a sensor for detecting a passenger status.

In some embodiments, the passenger information comprises health condition information and/or image information of a passenger, the health condition information comprising at least one of: blood pressure information, heart rate information, and pulse information, and the image information being collected by a camera pre-mounted on the driverless vehicle.

In some embodiments, the method further comprises: sending location information and/or vehicle identification information of the driverless vehicle to the cloud server.

According to a second aspect, the present application provides an apparatus for rescuing driverless vehicles, comprising: an information collecting unit, configured to collect status information of a driverless vehicle; a determining unit, configured to determine whether the driverless vehicle has an autonomous driving ability; a processing unit, configured to: set a vehicle driving mode of the driverless vehicle to a manual driving mode and further determine whether the driverless vehicle has a traveling ability in the manual driving mode, when the driverless vehicle does not have the autonomous driving ability; and an information sending unit, configured to collect passenger information of the driverless vehicle and send the status information and the passenger information to a cloud server providing support for the driverless vehicle, in response to the driverless vehicle not having the traveling ability in the manual driving mode.

In some embodiments, the determining unit is further configured to: determine that the driverless vehicle has the autonomous driving ability when following two conditions are both satisfied: the driverless vehicle has an ability of switching to an autonomous driving mode or the driverless vehicle is in the autonomous driving mode; and a sensor related to the autonomous driving mode and a circuit connected to the sensor are in a normal operating condition.

In some embodiments, the information collecting unit is further configured to: collect the status information of the driverless vehicle in real time or periodically.

In some embodiments, the sensor comprises at least one of: a sensor for detecting an operating status of a brake; a sensor for detecting an operating status of a vehicle-mounted battery; a camera, a sensor for detecting a fuel status; a sensor for detecting an air status inside the vehicle; and a sensor for detecting a passenger status.

In some embodiments, the passenger information comprises health condition information and/or image information of a passenger, the health condition information comprising at least one of:

blood pressure information, heart rate information, and pulse information, and the image information being collected by a camera pre-mounted on the driverless vehicle.

In some embodiments, the apparatus further comprises: an location information transmission unit, configured to send location information and/or vehicle identification information of the driverless vehicle to the cloud server.

According to the method and apparatus for rescuing driverless vehicles that are provided by the present application, it is determined whether a driverless vehicle has a traveling ability in an autonomous driving mode and in a manual driving mode; if the driverless vehicle does not have the traveling ability, status information of the driverless vehicle and information about passengers in the vehicle are collected and sent to a cloud server. In this way, it can be accurately determined whether the driverless vehicle has an autonomous driving ability, thereby efficiently rescuing driverless vehicles that break down.

### BRIEF DESCRIPTION OF THE DRAWINGS

Other features, objectives and advantages of the present application will become more apparent upon reading the detailed description to non-limiting embodiments with reference to the accompanying drawings, wherein:

FIG. 1 is an architectural diagram of a system in which the present application may be implemented;

FIG. 2 is a flow chart of a method for rescuing driverless vehicles according to an embodiment of the present application;

FIG. 3 is a schematic diagram of an application scenario of a method for rescuing driverless vehicles according to the present application;

FIG. 4 is a flow chart of a method for rescuing driverless vehicles according to another embodiment of the present application;

FIG. 5 is a schematic structural diagram of an apparatus for rescuing driverless vehicles according to an embodiment of the present application; and

FIG. 6 is a schematic structural diagram of a computer system adapted to implement a terminal device or a server according to the embodiments of the present application.

### DETAILED DESCRIPTION

The present application will be further described below in detail in combination with the accompanying drawings and the embodiments. It should be appreciated that the specific embodiments described herein are merely used for explaining the relevant disclosure, rather than limiting the disclosure. In addition, it should be noted that, for the ease of description, only the parts related to the relevant disclosure are shown in the accompanying drawings.

It should also be noted that the embodiments in the present application and the features in the embodiments may be combined with each other on a non-conflict basis. The present application will be described below in detail with reference to the accompanying drawings and in combination with the embodiments.

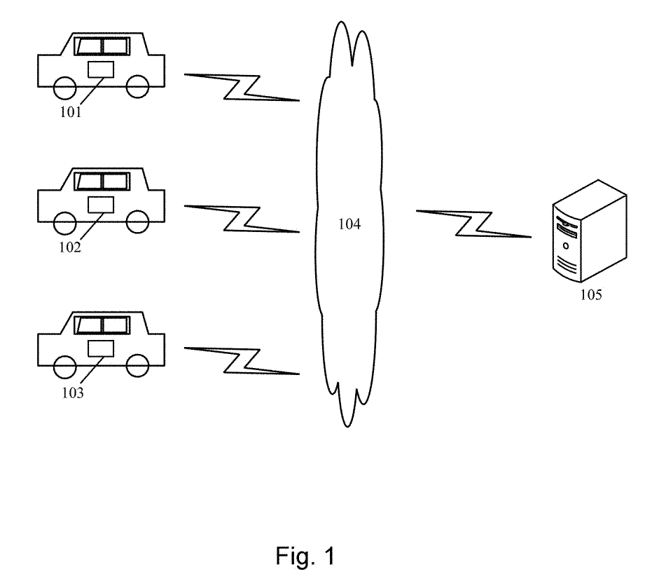

FIG. 1 shows a architecture of a system 100 which may be used by a method or apparatus for rescuing driverless vehicles according to an embodiment of the present application.

As shown in FIG. 1, the system architecture 100 may include vehicle-mounted terminal devices 101, 102 and 103, a network 104 and a cloud server 105 providing support for the vehicle-mounted terminal devices 101, 102 and 103. The network 104 serves as a medium providing a communication link between the vehicle-mounted terminal devices 101, 102 and 103 and the server 105. The network 104 may include various types of connections, such as wired or wireless transmission links, the global positioning system or optical fibers.

Driverless vehicle control systems may be installed on the vehicle-mounted terminal devices 101, 102 and 103. The vehicle-mounted terminal devices 101, 102 and 103 may directly interact with the cloud server 106 through the network 105. The vehicle-mounted terminal devices 101, 102 and 103 may be further connected to sensors for detecting the operating status of the vehicle, sensors for providing information input, displays, etc.

The server 105 may be a server providing rescue services, for example, a back-end cloud network server receiving distress signals sent by the vehicle-mounted terminal devices 101, 102 and 103 and providing rescue support. The back-end cloud network server may analyze and process data, such as, received condition information, passenger information, and make plans to implement rescues.

It should be noted that the method for rescuing driverless vehicles according to the embodiments of the present application is generally executed by the vehicle-mounted terminal devices 101, 102 and 103, and accordingly, an apparatus for rescuing driverless vehicles is generally provided in the vehicle-mounted terminal devices 101, 102 and 103.

It should be appreciated that the numbers of the terminal devices, the networks and the servers in FIG. 1 are merely illustrative. Any number of terminal devices, networks and servers may be provided based on the actual requirements.

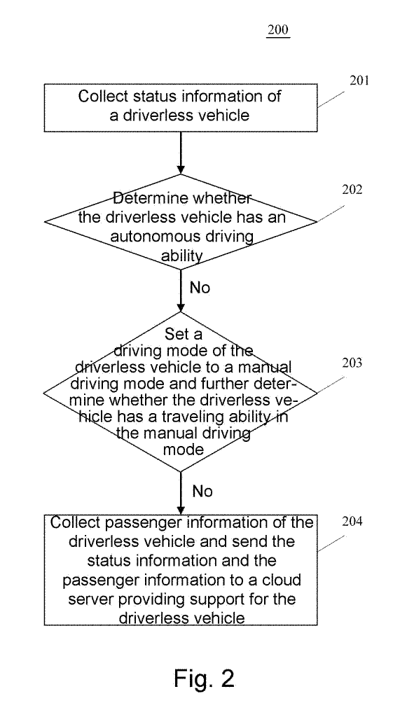

Further referring to FIG. 2, a flow 200 of a method for rescuing driverless vehicles according to an embodiment of the present application is shown. The method for rescuing driverless vehicles includes the following steps:

Step 201: Collect status information of a driverless vehicle.

In this embodiment, a vehicle-mounted terminal device (e.g., the vehicle-mounted terminal device 101, 102, and 103 shown in FIG. 1) on which a driverless vehicle control system is installed may collect data information of each sensor connected to the vehicle-mounted terminal device by means of data transmission. The above-mentioned data transmission includes, but is not limited to, parallel transmission, serial transmission, synchronous transmission, asynchronous transmission, simplex transmission, half-duplex transmission, and full-duplex transmission. The above-mentioned status information includes, but is not limited to, driving mode information and failure information.

Generally, the vehicle-mounted terminal device sends a data reading instruction to each sensor. After receiving the above-mentioned instruction, each sensor connected to the vehicle-mounted terminal device transmits its current data information to the vehicle-mounted terminal device. The vehicle-mounted terminal device receives the data information, and analyzes the collected data information to generate the status information. For example, after collecting information about the fuel amount, the vehicle-mounted terminal device compares the collected fuel amount with a fuel amount threshold predetermined by the vehicle-mounted terminal device, and if the collected fuel amount is below the predetermined fuel amount threshold, the vehicle-mounted terminal device generates status information related to the fuel amount and sends an alarm signal.

In some optional implementations of this embodiment, the status information in the above-mentioned method for rescuing driverless vehicles may be collected in real time or periodically. The above-mentioned collecting period may be set by a user, or may be set by default (e.g., the period is one second).

Step 202: Determine whether the driverless vehicle has an autonomous driving ability.

For example, the vehicle-mounted terminal device may directly determine that the above-mentioned driverless vehicle has the autonomous driving ability, when the driverless vehicle is currently traveling in the autonomous driving mode.

In some optional implementations of this embodiment, after determining that the above-mentioned driverless vehicle has the autonomous driving ability, the vehicle-mounted terminal device continues to execute step 201 to collect current vehicle status information of the driverless vehicle at a preset time interval, until it is determined that the driverless vehicle does not have the autonomous driving ability. The time interval may be set to 30 seconds, 10 minutes or the like. Taking 30 seconds as an example, after the vehicle-mounted terminal device determines that the driverless vehicle has the autonomous driving ability, the terminal device continuously collects status information at a time interval of 30 seconds, until it is determined that the driverless vehicle does not have the autonomous driving ability.

Step 203: Set a driving mode of the driverless vehicle to a manual driving mode and further determine whether the driverless vehicle has a traveling ability in the manual driving mode, when the driverless vehicle does not have the autonomous driving ability.

In this embodiment, when determining that the driverless vehicle does not have the autonomous driving ability at step 202, the above-mentioned vehicle-mounted terminal device sets the vehicle driving mode of the driverless vehicle to the manual driving mode. After analyzing the driving ability, the vehicle-mounted terminal device sends mode setting instruction information to a vehicle mode setting system. Specifically, if the current driving mode of the vehicle is the autonomous driving mode, the vehicle-mounted terminal device sends an instruction of switching to the manual driving mode to the vehicle mode setting system. After receiving the instruction, the vehicle mode setting system executes the instruction to perform a switching action. If the current driving mode of the vehicle is the manual driving mode, the vehicle-mounted terminal device sends an instruction of maintaining this state to the vehicle mode setting system. After receiving the instruction, the vehicle mode setting system executes the instruction of maintaining this state.

In this embodiment, after setting the driving mode of the driverless vehicle to the manual driving mode, the vehicle-mounted terminal device further determines whether the driverless vehicle has a traveling ability in this mode. Specifically, in the manual driving mode, the vehicle-mounted terminal device collects data information related to manual driving, the data information including, but not limited to, steering wheel information, braking information, tire pressure information, and fuel amount information. For example, when the brake or the steering wheel of the driverless vehicle malfunction in the manual mode, the driver inside the above-mentioned driverless vehicle enables an emergency rescue function. After receiving the emergency rescue information, the vehicle-mounted terminal device collects the above-mentioned information related to the manual driving mode, and starts a program of forcing the vehicle to stop.

Step 204: Collect passenger information of the driverless vehicle and send the status information and the passenger information to a cloud server providing support for the driverless vehicle, if the driverless vehicle does not have the traveling ability in the manual driving mode.

In this embodiment, if it is determined at step 203 that the driverless vehicle does not have the traveling ability in the manual driving mode, information about the passengers in the driverless vehicle is collected, and the collected information about the passengers in the vehicle and the status information collected at step 201 are sent to a cloud server that provides support for the driverless vehicle.

In some optional implementations of this embodiment, the above-mentioned information about the passenger in the vehicle includes, but is not limited to, health information and image information of the passenger in the vehicle. A sensor for collecting health information of a human body, for example, a sensor for measuring the blood pressure and a sensor for measuring the heart rate, is pre-provided in the driverless vehicle. The vehicle-mounted terminal device collects the health information of the human body by collecting information from the sensor related to human health. The health information includes, but is not limited to, blood pressure information, heart rate information, and pulse information. A movable camera may be pre-provided in the driverless vehicle, to shoot pictures of the seat of the passenger in the vehicle, and convert the pictures to an image form so as to transmit to the vehicle-mounted terminal device.

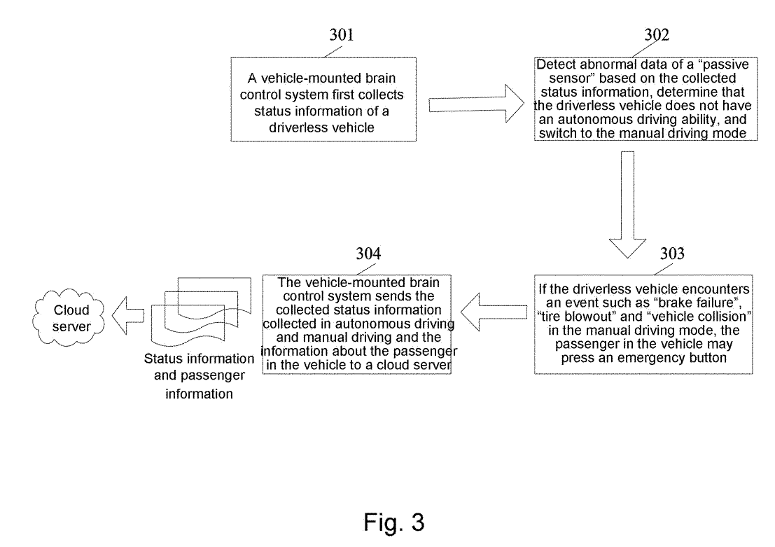

Further referring to FIG. 3, a schematic diagram of an application scenario of the method for rescuing driverless vehicles according to this embodiment is shown. In the application scenario of FIG. 3, the vehicle-mounted terminal device collects current status information of the driverless vehicle first, and then analyzes the collected status information to check whether the driverless vehicle is able to travel in the autonomous driving mode. For example, if the vehicle-mounted terminal device detects abnormal data of a “passive sensor” in the autonomous driving mode, indicating that the driverless vehicle cannot accurately detect the position of an obstacle, the vehicle-mounted terminal device determines that the driverless vehicle does not have the autonomous driving ability, and therefore switches the driving mode of the vehicle to the manual driving mode. If the driverless vehicle encounters an event such as “brake failure”, “tire blowout” and “vehicle collision” in the manual driving mode, the passenger in the vehicle may press an emergency button that is pre-provided in the vehicle. After receiving the emergency information, the vehicle-mounted terminal device sends the vehicle status information collected in autonomous driving and manual driving and the information about the passenger in the vehicle to the cloud server.

By collecting the status information and accurately determining whether the driverless vehicle has the traveling ability, the method provided by the above-mentioned embodiment of the present application can thereby efficiently rescuing driverless vehicles that break down.

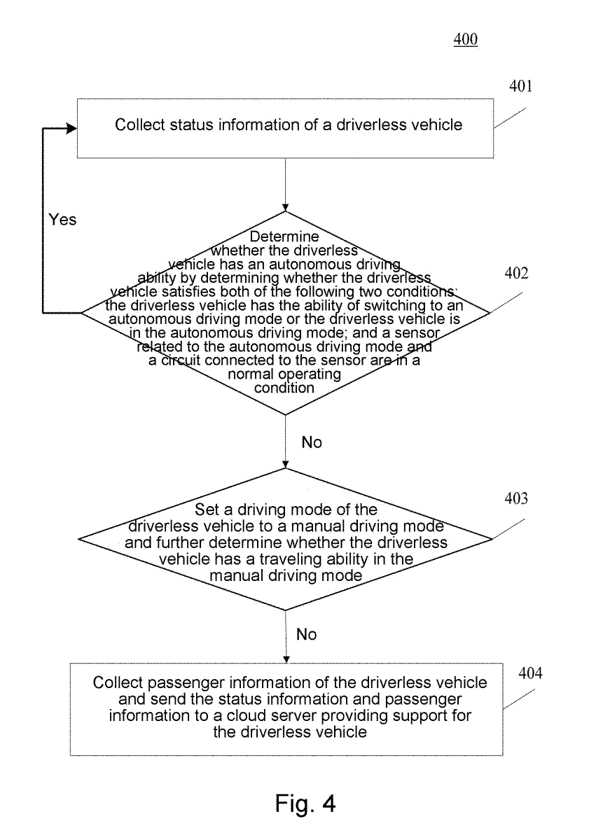

Further referring to FIG. 4, a flow 400 of a method for rescuing driverless vehicles according to another embodiment is shown. The flow 400 of the method for rescuing driverless vehicles includes the following steps:

Step 401: Collect status information of a driverless vehicle.

In this embodiment, the concrete process of step 401 can refer to step 201 in the embodiment corresponding to FIG. 2, and will not be repeated herein.

Step 402: Determine whether the driverless vehicle has an autonomous driving ability by determining whether the driverless vehicle satisfies both of the following two conditions: the driverless vehicle has the ability of switching to an autonomous driving mode or the driverless vehicle is in the autonomous driving mode; and a sensor related to the autonomous driving mode and a circuit connected to the sensor are in a normal operating condition.

In this embodiment, based on the status information received at step 401, it is detected whether the vehicle driving mode of the driverless vehicle is the autonomous driving mode. If yes, this mode is maintained; otherwise, it is detected whether the above-mentioned driverless vehicle has the ability of switching to the autonomous driving mode. If the above-mentioned driverless vehicle has the ability of switching to the autonomous driving mode, the driving mode of the above-mentioned vehicle is switched to the autonomous driving mode; otherwise, it is determined that the driverless vehicle does not have a driving ability.

In this embodiment, when the above-mentioned driverless vehicle is in the autonomous driving mode, the above-mentioned status information is further checked to determine whether the sensor related to the autonomous driving mode and the circuit connected to the sensor are in the normal operating condition. Determining whether the sensor related to the autonomous driving mode and the circuit connected to the sensor are in the normal operating condition includes: sending, by the vehicle-mounted terminal device, instruction information to the above-mentioned sensor and monitoring feedback information from the above-mentioned sensor; if the vehicle-mounted terminal device does not detect any feedback information, indicating that a failure occurs in the above-mentioned sensor and/or the circuit connected to the sensor, determining that the driverless vehicle does not have the autonomous driving ability; if the vehicle-mounted terminal device detects feedback information, further analyzing the feedback information to determine whether a failure occurs in the above-mentioned sensor. If a failure occurs in the sensor, the driverless vehicle does not have the autonomous driving ability; if no failure occurs in the sensor, the driverless vehicle has the autonomous driving ability. The sensor related to the autonomous driving mode includes, but is not limited to, a sensor for detecting an operating status of a brake, a sensor for detecting an operating status of a vehicle-mounted battery, a camera, a sensor for detecting a fuel status, a sensor for detecting an air status inside the vehicle, and a sensor for detecting a passenger status.

In this embodiment, if the driverless vehicle has the autonomous driving ability, the vehicle-mounted terminal device continues to execute step 201 to collect current vehicle status information of the driverless vehicle at a predetermined time interval, until it is determined that the driverless vehicle does not have the autonomous driving ability. If the driverless vehicle does not have the autonomous driving ability, step 403 is executed.

Step 403: Set a driving mode of the driverless vehicle to a manual driving mode and further determine whether the driverless vehicle has a traveling ability in the manual driving mode, when the driverless vehicle does not have the autonomous driving ability.

In this embodiment, the concrete process of step 403 can refer to step 203 in the embodiment corresponding to FIG. 2, and will not be repeated herein.

Step 404: Collect passenger information of the driverless vehicle and send the status information and passenger information to a cloud server providing support for the driverless vehicle, when the driverless vehicle does not have the traveling ability in the manual mode.

In this embodiment, based on the above-mentioned step 402 where it is determined whether the driverless vehicle has the traveling ability in the manual driving mode, if the driverless vehicle does not have the traveling ability in the manual driving mode, information about the passenger in the driverless vehicle is collected, and the collected information about the passenger and the above-mentioned status information obtained in step 401 are sent to the cloud server.

In some optional implementations of this embodiment, the information sent to the cloud server providing support for the above-mentioned driverless vehicle further includes location information and identification information of the above-mentioned driverless vehicle. The location information may include, but is not limited to, ambient environment information and current road information. The identification information of the driverless vehicle includes, but is not limited to, license plate number information, model information, and color information.

As can be seen from FIG. 4, the main difference between this embodiment and the embodiment corresponding to FIG. 2 lies in that the flow 400 of the method for rescuing driverless vehicles in this embodiment highlights step 402 where it is determined whether the driverless vehicle has the autonomous driving ability, by determining whether the driverless vehicle satisfies two conditions: the driverless vehicle has the ability of switching to the autonomous driving mode or the driverless vehicle is in the autonomous driving mode; and the sensor related to the autonomous driving mode and the circuit connected to the sensor are in the normal operating condition. By means of step 402 which is an improvement on step 202, the solution described in this embodiment is more targeted, and can more accurately determine whether the driverless vehicle has the autonomous driving ability.

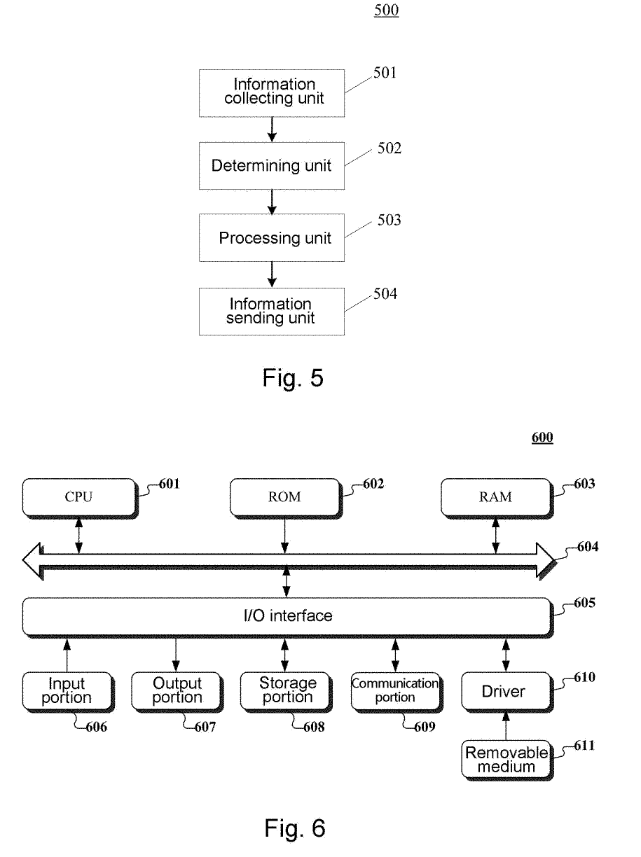

Further referring to FIG. 5, as an implementation of the methods as shown in the above-mentioned figures, the present application provides an embodiment for an apparatus for rescuing driverless vehicles. This apparatus embodiment corresponds to the method embodiment shown in FIG. 2. The apparatus may be specifically applied to a vehicle-mounted terminal device of a driverless vehicle.

As shown in FIG. 5, the apparatus 500 for rescuing driverless vehicles according to this embodiment includes: an information collecting unit 501, a determining unit 502, a processing unit 503, and an information sending unit 504. The information collecting unit 501 is configured to collect status information of a driverless vehicle. The determining unit 502 is configured to determine whether the driverless vehicle has an autonomous driving ability. The processing unit 503 is configured to: set a vehicle driving mode of the driverless vehicle to a manual driving mode and further determine whether the driverless vehicle has a traveling ability in the manual driving mode, when the driverless vehicle does not have the autonomous driving ability. The information sending unit 504 is configured to collect passenger information of the driverless vehicle and sending the vehicle status information and the passenger information to a cloud server that provides support for the driverless vehicle, in response to the driverless vehicle not having the traveling ability in the manual driving mode.

In this embodiment, the information collecting unit 501 in the apparatus 500 for rescuing driverless vehicles may collect data information of each sensor connected to the vehicle-mounted terminal device by means of data transmission. The above-mentioned collected vehicle status information includes, but is not limited to, driving mode information, failure information, fuel amount detection information, tire pressure information, detection information about the ambient environment, and information about operation of each sensor. The above-mentioned vehicle status information includes, but is not limited to, driving mode information and failure information.

In this embodiment, based on the vehicle status information collected by the information collecting unit 501, the above-mentioned determining unit 502 may analyze the status information to determine whether the driverless vehicle has an autonomous driving ability in the current state. If not, the processing unit 503 is triggered.

In this embodiment, if the determining unit 502 determines that the driverless vehicle does not have the autonomous driving ability, the above-mentioned processing unit 503 sends mode setting instruction information to a vehicle mode setting system after analyzing the driving ability, to set the driving mode to the manual driving mode; and after setting the driving mode of the driverless vehicle to the manual driving mode, further determines whether the driverless vehicle has a traveling ability in this mode based on the collected data information related to the manual driving mode. If not, the information sending unit 504 is triggered.

In this embodiment, if the determining unit 502 determines that the driverless vehicle does not have a traveling ability in the manual driving mode, the above-mentioned information sending unit 504 collects information about a passenger in the driverless vehicle, and sends the collected information about the passenger and the vehicle status information in the information collecting unit 501 to the cloud server.

In some optional implementations of this embodiment, the determining unit 502 in the above-mentioned apparatus 500 for rescuing driverless vehicles is further configured to determine whether the driverless vehicle has an autonomous driving ability by detecting whether the driverless vehicle satisfies both of the following two conditions: the driverless vehicle has the ability of switching to an autonomous driving mode or the driverless vehicle is in the autonomous driving mode; and a sensor related to the autonomous driving mode and a circuit connected to the sensor are in a normal operating condition. Optionally, the above-mentioned sensor may include at least one of: a sensor for detecting an operating status of a brake; a sensor for detecting an operating status of a vehicle-mounted battery; a camera; a sensor for detecting a fuel status; a sensor for detecting an air status inside the vehicle; and a sensor for detecting a passenger status.

In some optional implementations of this embodiment, the above-mentioned apparatus 500 for rescuing driverless vehicles includes a location information transmission unit (not shown), configured to collect the status information of the driverless vehicle in real time or periodically. The above-mentioned collecting period may be set by a user, or may be set by default (e.g., the period is one second).

In some optional implementations of this embodiment, the information sent by the information sending unit in the above-mentioned apparatus 500 for rescuing driverless vehicles to the above-mentioned cloud server further includes location information and identification information of the above-mentioned driverless vehicle. The location information may include, but is not limited to, ambient environment information and current road information. The identification information of the driverless vehicle includes, but is not limited to, license plate number information, model information, and color information.

In some optional implementations of this embodiment, the passenger information collected by the information sending unit in the above-mentioned apparatus 500 for rescuing driverless vehicles may further include health information and/or image information of a passenger. A sensor for collecting health information of a human body, for example, a sensor for measuring the blood pressure and a sensor for measuring the heart rate, is pre-provided in the driverless vehicle. The vehicle-mounted terminal device collects the health information of the human body by collecting information from the sensor related to human health. The health information includes, but is not limited to, blood pressure information, heart rate information, and pulse information. A movable camera may be pre-provided in the driverless vehicle, to shoot pictures of the seat of the passenger in the vehicle, and convert the pictures to an image form so as to transmit the vehicle-mounted terminal device.

Referring to FIG. 6, a schematic structural diagram of a computer system 600 adapted to implement a terminal device or a server of the embodiments of the present application is shown.

As shown in FIG. 6, the computer system 600 includes a central processing unit (CPU) 601, which may execute various appropriate actions and processes in accordance with a program stored in a read-only memory (ROM) 602 or a program loaded into a random access memory (RAM) 603 from a storage portion 608. The RAM 603 also stores various programs and data required by operations of the system 600. The CPU 601, the ROM 602 and the RAM 603 are connected to each other through a bus 604. An input/output (I/O) interface 605 is also connected to the bus 604.

The following components are connected to the I/O interface 605: an input portion 606 including a keyboard, a mouse etc.; an output portion 607 comprising a liquid crystal display device (LCD), a speaker etc.; a storage portion 608 including a hard disk and the like; and a communication portion 609 comprising a network interface card, such as a LAN card and a modem. The communication portion 609 performs communication processes via a network, such as the Internet. A driver 610 is also connected to the I/O interface 605 as required. A removable medium 611, such as a magnetic disk, an optical disk, a magneto-optical disk, and a semiconductor memory, may be installed on the driver 610, to facilitate the retrieval of a computer program from the removable medium 611, and the installation thereof on the storage portion 608 as needed.

In particular, according to an embodiment of the present disclosure, the process described above with reference to the flowcharts may be implemented in a computer software program. For example, an embodiment of the present disclosure includes a computer program product, which comprises a computer program that is tangibly embedded in a machine-readable medium. The computer program comprises program codes for executing the method of the flowcharts. In such an embodiment, the computer program may be downloaded and installed from a network via the communication portion 609, and/or may be installed from the removable media 611.

The flowcharts and block diagrams in the figures illustrate architectures, functions and operations that may be implemented according to the system, the method and the computer program product of the various embodiments of the present disclosure.

In this regard, each block in the flowcharts and block diagrams may represent a module, a program segment, or a code portion. The module, the program segment, or the code portion comprises one or more executable instructions for implementing the specified logical function. It should be noted that, in some alternative implementations, the functions denoted by the blocks may occur in a sequence different from the sequences shown in the figures. For example, in practice, two blocks in succession may be executed, depending on the involved functionalities, substantially in parallel, or in a reverse sequence. It should also be noted that, each block in the block diagrams and/or the flow charts and/or a combination of the blocks may be implemented by a dedicated hardware-based system executing specific functions or operations, or by a combination of a dedicated hardware and computer instructions.

The units involved in the embodiments of the present application may be implemented byway of software or hardware. The described units may also be provided in a processor, for example, described as: a processor, comprising an information collecting unit, a determining unit, a processing unit and an information sending unit, where the names of these units or modules are not considered as a limitation to the units or modules. For example, the information collecting unit may also be described as “a unit for collecting status information of a driverless vehicle”.

In another aspect, the present application further provides a computer readable storage medium. The computer readable storage medium may be the computer readable storage medium included in the apparatus in the above embodiments, or a stand-alone computer readable storage medium which has not been assembled into the apparatus. The computer readable storage medium stores one or more programs. The one or more programs, when executed by a device, cause the device to: collect status information of a driverless vehicle; determine whether the driverless vehicle has an autonomous driving ability; if not, set a driving mode of the driverless vehicle to a manual driving mode and further determining whether the driverless vehicle has a traveling ability in the manual driving mode; and collect passenger information of the driverless vehicle and sending the status information and the passenger information to a cloud server providing support for the driverless vehicle, in response to the driverless vehicle not having the traveling ability in the manual driving mode.

The foregoing is only a description of the embodiments of the present application and the applied technical principles. It should be appreciated by those skilled in the art that the inventive scope of the present application is not limited to the technical solutions formed by the particular combinations of the above technical features. The inventive scope should also cover other technical solutions formed by any combinations of the above technical features or equivalent features thereof without departing from the concept of the disclosure, such as, technical solutions formed by replacing the features as disclosed in the present disclosure with (but not limited to), technical features with similar functions.

What is claimed is:

1. A method for rescuing driverless vehicles, comprising:
collecting status information of a driverless vehicle;
determining whether the driverless vehicle has an autonomous driving ability;
if not, setting a driving mode of the driverless vehicle to a manual driving mode and further determining whether the driverless vehicle has a traveling ability in the manual driving mode; and
collecting passenger information of the driverless vehicle and sending the status information and the passenger information to a cloud server providing support for the driverless vehicle, in response to the driverless vehicle not having the traveling ability in the manual driving mode.

2. The method according to claim 1, wherein the determining whether the driverless vehicle has the autonomous driving ability comprises:
determining that the driverless vehicle has the autonomous driving ability when following two conditions are both satisfied: the driverless vehicle has an ability of switching to an autonomous driving mode or the driverless vehicle is in the autonomous driving mode; and a sensor related to the autonomous driving mode and a circuit connected to the sensor are in a normal operating condition.

3. The method according to claim 1, wherein the collecting status information of the driverless vehicle comprises:
collecting the status information of the driverless vehicle in real time or periodically.

4. The method according to claim 2, wherein the sensor comprises at least one of:
a sensor for detecting an operating status of a brake;
a sensor for detecting an operating status of a vehicle-mounted battery;
a camera;
a sensor for detecting a fuel status;
a sensor for detecting an air status inside the vehicle; and
a sensor for detecting a passenger status.

5. The method according to claim 1, wherein the passenger information comprises health condition information and/or image information of a passenger, the health condition information comprising at least one of: blood pressure information, heart rate information, and pulse information, and the image information being collected by a camera pre-mounted on the driverless vehicle.

6. The method according to claim 1, wherein the method further comprises: sending location information and/or vehicle identification information of the driverless vehicle to the cloud server.

7. A system for rescuing driverless vehicles, comprising:
a processor; and
a memory coupled to the processor and storing computer readable instructions which when executed by the processor, cause the processor to perform a method for rescuing driverless vehicles, the method comprising:
collecting status information of a driverless vehicle;
determining whether the driverless vehicle has an autonomous driving ability;
if not, setting a driving mode of the driverless vehicle to a manual driving mode and further determining whether the driverless vehicle has a traveling ability in the manual driving mode; and
collecting passenger information of the driverless vehicle and sending the status information and the passenger information to a cloud server providing support for the driverless vehicle, in response to the driverless vehicle not having the traveling ability in the manual driving mode.

8. The system according to claim 7, wherein the determining whether the driverless vehicle has the autonomous driving ability comprises:
determining that the driverless vehicle has the autonomous driving ability when following two conditions are both satisfied: the driverless vehicle has an ability of switching to an autonomous driving mode or the driverless vehicle is in the autonomous driving mode; and a sensor related to the autonomous driving mode and a circuit connected to the sensor are in a normal operating condition.

9. The system according to claim 7, wherein the collecting status information of the driverless vehicle comprises:
collecting the status information of the driverless vehicle in real time or periodically.

10. The system according to claim 8, wherein the sensor comprises at least one of:
a sensor for detecting an operating status of a brake;
a sensor for detecting an operating status of a vehicle-mounted battery;
a camera;
a sensor for detecting a fuel status;
a sensor for detecting an air status inside the vehicle; and
a sensor for detecting a passenger status.

11. The system according to claim 7, wherein the passenger information comprises health condition information and/or image information of a passenger, the health condition information comprising at least one of: blood pressure information, heart rate information, and pulse information, and the image information being collected by a camera pre-mounted on the driverless vehicle.

12. The system according to claim 7, wherein the method further comprises: sending location information and/or vehicle identification information of the driverless vehicle to the cloud server.

13. Anon-transitory storage medium storing one or more programs, the one or more programs when executed by an apparatus, causing the apparatus to perform a method for rescuing driverless vehicles, the method comprising:
collecting status information of a driverless vehicle;
determining whether the driverless vehicle has an autonomous driving ability;
if not, setting a driving mode of the driverless vehicle to a manual driving mode and further determining whether the driverless vehicle has a traveling ability in the manual driving mode; and
collecting passenger information of the driverless vehicle and sending the status information and the passenger information to a cloud server providing support for the driverless vehicle, in response to the driverless vehicle not having the traveling ability in the manual driving mode.
